(19) United States
(12) Patent Application Publication
(10) Pub. No.: US 2023/0251203 A1
(43) Pub. Date: Aug. 10, 2023

(54) METHOD FOR EVALUATING ADVERSE REACTION OF SESQUITERPENOIDS IN ZEDOARY TURMERIC OIL

(71) Applicant: BEIJING FRIENDSHIP HOSPITAL, CAPITAL MEDICAL UNIVERSITY, Beijing (CN)

(72) Inventors: Dan YAN, Beijing (CN); Yan YAN, Beijing (CN); Xiaofang WANG, Beijing (CN); Zhenzhen WANG, Beijing (CN)

(73) Assignee: BEIJING FRIENDSHIP HOSPITAL, CAPITAL MEDICAL UNIVERSITY, Beijing (CN)

(21) Appl. No.: 17/623,235

(22) PCT Filed: Apr. 26, 2021
(86) PCT No.: PCT/CN2021/089757

§ 371 Date: Dec. 27, 2021

(30) Foreign Application Priority Data
Feb. 3, 2021 (CN) 202110144114.3

(51) Int. Cl. G01N 21/75; G01N 21/31; G01N 33/72

(52) U.S. Cl. CPC G01N 21/75; G01N 21/31; G01N 33/721

(57) ABSTRACT

A method for evaluating adverse reaction of sesquiterpenoids in zedoary turmeric oil is performed successively according to the following steps: (1) preparing a hemoglobin (Hb) solution and a to-be-determined solution; (2) taking Hb solutions with a same volume and respectively adding the same volume of the to-be-determined solution or normal saline thereto, mixing well and standing; (3) determining absorbance with a microplate reader, and comparing the absorbance of a to-be-determined solution group with the absorbance of a normal saline group to obtain a value r; wherein if r>1.5, then the result indicates that the concentration of the to-be-determined solution has a risk of causing dyspnea; wherein,

$$r = \frac{OD}{{OD}_{Hb}} ;$$

in the formula, OD is an absorbance at 280 nm wavelength of the to-be-determined solution after interacting with Hb; OD_{Hb}, is an absorbance at 280 nm wavelength of a blank control of normal saline after interacting with Hb.

### CROSS REFERENCE TO THE RELATED APPLICATIONS

This application is the national phase entry of International Application No. PCT/CN2021/089757, filed on Apr. 26, 2021, which is based upon and claims priority to Chinese Patent Application No. 202110144114.3, filed on Feb. 3, 2021, the entire contents of which are incorporated herein by reference.

### TECHNICAL FIELD

The present invention relates to the field of pharmaceutical determination, and in particular to a method for evaluating adverse reaction of sesquiterpenoids in zedoary turmeric oil.

### BACKGROUND

Zedoary turmeric oil is a kind of volatile oil component extracted from a traditional Chinese medicine Rhizomacurcumae (dry rhizomes of Curcuma phaeocaulis Val., Curcuma kwangsiensis S. G. Lee et C. F. Liang, or Curcuma wenyujin Y H. Chen et C. Ling). Existing related researches have proved that zedoary turmeric oil has the effects of immune activation, anticancer, anti-inflammation, antiviral action, and the like. According to different therapeutic goals, the clinically applied dosage forms include suppositories, ointments, injections and the like. Major chemical components in zedoary turmeric oil are sesquiterpenoids, such as, curzerenone, curcumenol and elemene. Adverse reaction conditions of related formulations containing zedoary turmeric oil were issued in the Adverse Drug Reaction Reporting of the National Medical Products Administration (No. 7) about adverse drug reaction of zedoary turmeric oil injection on Dec. 17, 2004; dyspnea is one of the adverse reaction. Analysis on 81 Cases of Adverse Reaction of Zedoary Turmeric Oil published in Chinese Pharmaceutical Affairs in 2007 has reported that the adverse reaction caused by zedoary turmeric oil is clinically manifested by labored breathing, dyspnea, and the like.

These literature reports only perform analysis on the clinical adverse reaction phenomenon of zedoary turmeric oil, but neither establish the corresponding evaluation method to the early safety warning in clinical application, nor make clear the material basis causing dyspnea (namely, do not explain what kind of ingredient in zedoary turmeric oil causes dyspnea), which shows that the problem has been still not clinically solved effectively and early warning has been not achieved.

The adverse reaction cases caused by elemene injection have been analyzed by Serial Analysis on 7 Severe Adverse Drug Reaction Cases Due to Intrathoracic Injection of Elemene published in Chinese Journal of Lung Cancer in 2018 to find that the adverse drug reaction of Elemene Injection is mainly manifested by dyspnea, and asthmatic symptom, which is similar to the adverse reaction of zedoary turmeric oil. But the above research still only represents or describes the related clinical adverse reaction phenotype, and neither establishes the corresponding evaluation method to the early safety warning in clinical application, nor explains the mechanism of action or process caused by the adverse reaction thereof. Chinese patent CN103242275A discloses that a composition of guaiane Drug-type and Eucalyptus-type sesquiterpenoids in Rhizomacurcumae may be used as pathogenesis for the treatment of tumors, inflammation, immune and other diseases correlated to NO metabolic disorder. The patent only introduces the pharmacological activity of sesquiterpenoids in partial zedoary turmeric oil, but does not relate to a phenomenon of causing adverse reaction of dyspnea.

In view of this, this present invention is provided herein.

### SUMMARY

The present invention discloses a method for evaluating adverse reaction of sesquiterpenoids in zedoary turmeric oil, which provides early warning for the clinical safety medication of medicaments or related formulations with sesquiterpenoids in zedoary turmeric oil as major components. The method is performed successively according to the following steps:
(1) preparing a hemoglobin (Hb) solution and a to-be-determined solution;
(2) taking Hb solutions with a same volume and respectively adding the same volume of the to-be-determined solution or normal saline thereto, mixing well and standing;
(3) determining absorbance with a microplate reader, and comparing the absorbance of a to-be-determined solution group with the absorbance of a normal saline group to obtain a value r; where if r>1.5, then the result indicates that the concentration of the to-be-determined solution has a risk of causing dyspnea; wherein,

$$r = \frac{OD}{{OD}_{Hb}} ,$$

in the formula, OD is an absorbance value at a wavelength of 280 nm of the to-be-determined solution after interacting with Hb; OD_{Hb}, is an absorbance value at a wavelength of 280 nm of the normal saline after interacting with Hb (as a blank control).

Preferably, in the step (1), the prepared Hb solution has a concentration of 2 mg/mL.

Preferably, in the step (2), the to-be-determined solution and the normal saline used have a volume of 100 μL.

Preferably, the added Hb solution has a volume of 100 μL.

Preferably, the step of mixing well in the step (2) is performed by using an oscillator for oscillation for 5 s.

Preferably, the standing in the step (2) is performed for 10 min at a condition of 25° C.

Preferably, conditions for the absorbance determination in the step (3) are as follows: performing spectrum scanning at 37° C. and 230-400 nm with a step size of 5 nm.

Compared with the prior art, the present invention has the following advantages:

The present invention discloses that sesquiterpenoids in zedoary turmeric oil can bind to hemoglobin and cause the helix removal of hemoglobin α-helix to decrease the oxygen-loading amount of hemoglobin, thus causing the occurrence of dyspnea. The present invention further discloses a method for evaluating adverse reaction of sesquiterpenoids in zedoary turmeric oil, which provides early warning for the clinical safety medication of medicaments or related formulations containing sesquiterpenoids in zedoary turmeric oil.

### BRIEF DESCRIPTION OF THE DRAWINGS

FIG. 1 shows a structural formula of a sesquiterpenoid β-elemene (BE) in zedoary turmeric oil;

FIG. 2 is a graphical result showing the influences of β-elemene on partial pressure of arterial oxygen of rats;

FIG. 3 is a graphical result showing the influences of β-elemene on arterial partial pressure of carbon dioxide of rats;

FIG. 4 is a graphical result showing the influences of β-elemene on arterial blood pH of rats;

FIG. 5 is a graphical result showing the influences of β-elemene on arterial blood oxygen saturation of rats;

FIG. 6 is a graphical result showing the influences of β-elemene on total hemoglobin of rats;

FIG. 7 is a graphical result showing the influences of β-elemene on oxyhemoglobin of rats;

FIG. 8 is a graphical result showing the interaction between β-elemene and hemoglobin;

FIG. 9 is a graphical fitting result showing the interaction between β-elemene and hemoglobin;

FIG. 10 is a graphical result showing the influences of β-elemene on a hemoglobin structure;

FIG. 11 is a graphical result showing the influences of β-elemene on UV absorbance of hemoglobin.

In the drawing, * represents as follows: *p<0.05; **p<0.01; ***p<0.001; ****p<0.0001.

### DETAILED DESCRIPTION OF THE EMBODIMENTS

To further explain the technical means and results taken in this present invention to achieve the predetermined goals of the present invention, β-elemene in zedoary turmeric oil is set as an example in this present invention. The following preferred examples are used to describe the specific embodiments, technical solutions and features based on the present invention application hereafter. Specific features, structures, or characteristics of the multiple examples in the following description can be combined in any suitable form.

Main materials and sources selected and used in the following examples of the present invention are respectively as follows:

The sesquiterpenoid in zedoary turmeric oil, β-elemene (1S, 2S, 4R)-1-vinyl-methyl-2, 4-di(1-methylvinyl) cyclohexane, BE, has a structural formula shown in FIG. 1, has a purity>98%, and is purchased from CHENGDU MUST BIO-TECHNOLOGY CO., LTD, CAS: 33880-83-0); quercetin, 3,3′,4′,5,7-pentahydroxyflavone, has a purity≥98%, and is purchased from CHENGDU MUST BIO-TECHNOLOGY CO., LTD, CAS: 117-39-5); hemoglobin, Hb (purchased from Sigma, Art. No.: H7379); normal saline (Beijing Shuanghe Pharmaceutical Co. Ltd.); surgical instruments and G3+blood-gas test strip (Beijing Bio-Asia Technology and Trade Co., Ltd.); Abbotti-STAT300 portable hand-hold blood-gas analyzer (i-STAT); BL-420E+biological functioning experiment system (Chengdu Taimeng Technology Co., Ltd.); chloral hydrate (Dalian Meilun Biotech Co., Ltd); 96-well plate (Corning); microfiltration membrane (0.22 μm, Sartorius Stedim Biotech); 1.5 mL/50 mL/10 mL centrifugal tube (Corning); DMSO (D8418, Sigma); Tween 80 (Sigma); PBS-P+10× (GE healthcare); vortex mixer (Vortex Genie 2, Scientific Industries); microplate reader (Bio-Tek); microwell plate thermostatic oscillator (MB100-4A); CMS sensor chip (U.S. BR100530); ultrapure water machine (Milli-Q Integral 5.5 Kit); surface plasma resonance spectrometer (U.S. Biacore T2000); and CD spectrometer (Britain Chirascan).

### Example I: Influences of β-Elemene (BE) on Blood Gas when Causes Dyspnea of Rats

Preparation of a BE solution: BE reference substances were precisely weighed and dissolved by 8.8% Tween 80 solution and prepared into a BE solution having a concentration of 1.5 mg/mL.

12 pieces of SPF-grade SD male rats (6 weeks of age, weight: 200±25 g) were taken; and these rats were purchased from Beijing Vital River Laboratory Animal Technology Co., Ltd, production license number: SCXK (J.) 2016-0006.Animal experiments involved in the research process were approved by Ethics Committee. The feeding conditions of the experimental rats are as follows: standard illumination-dark period is 12 h, temperature is 22±1° C., and relative humidity is 60±5%.

The rats were randomly divided into 2 groups, 6 pieces each group. The rats were adaptively fed with common fodders for one week. Then the rats were subjected to overnight fasting (12 h), and then intraperitoneally injected with chloral hydrate (10 mg/mL, 4 mL/kg) for anesthetization. After anesthetization, the two groups were respectively injected with BE and normal saline via caudal vein with an administration volume of 6 mL/kg.

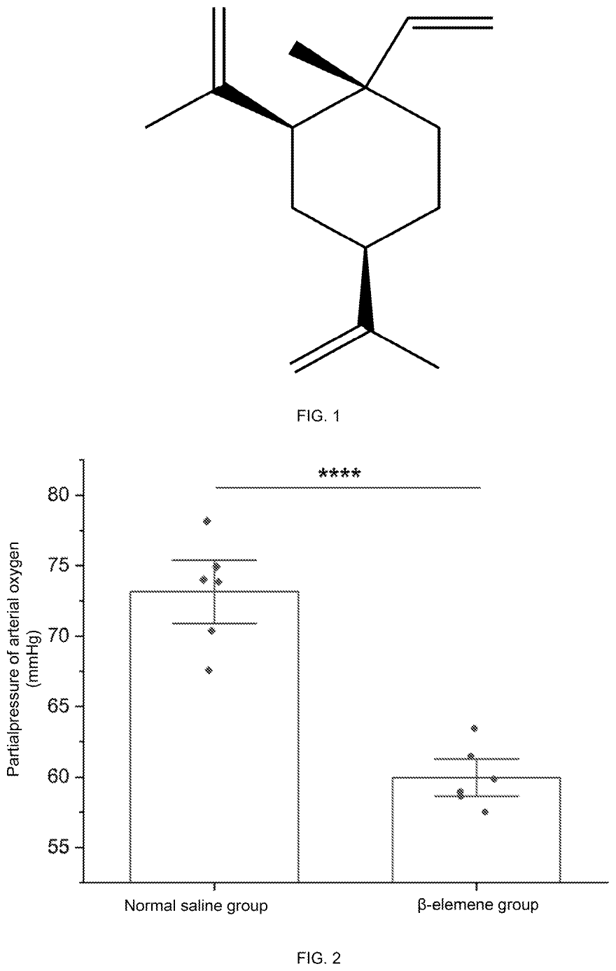

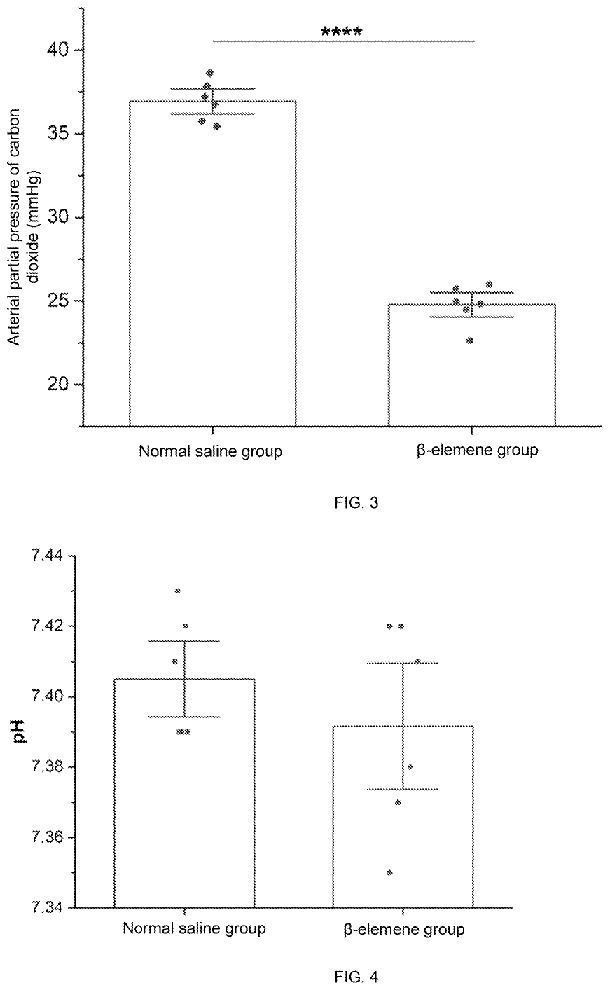

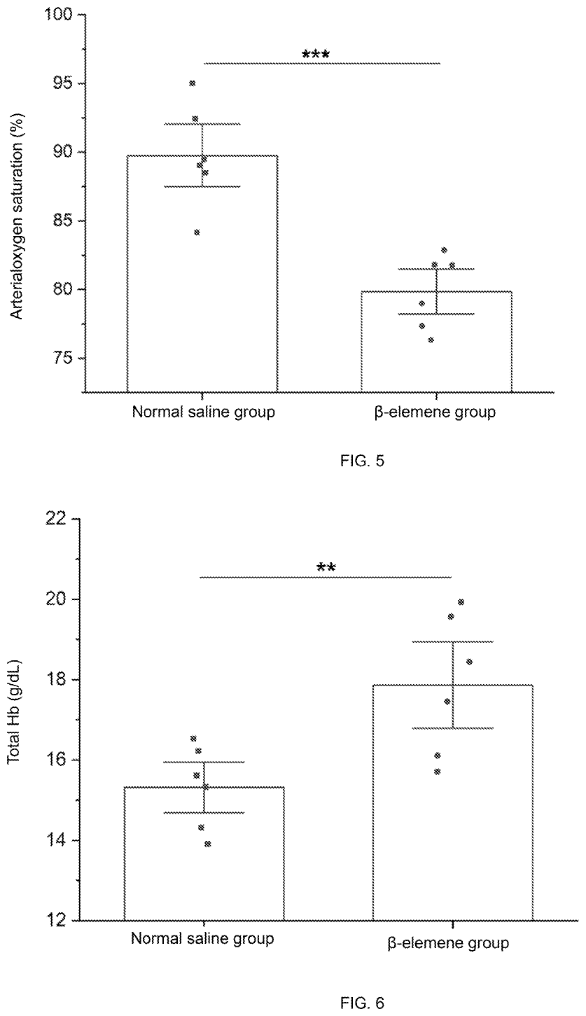

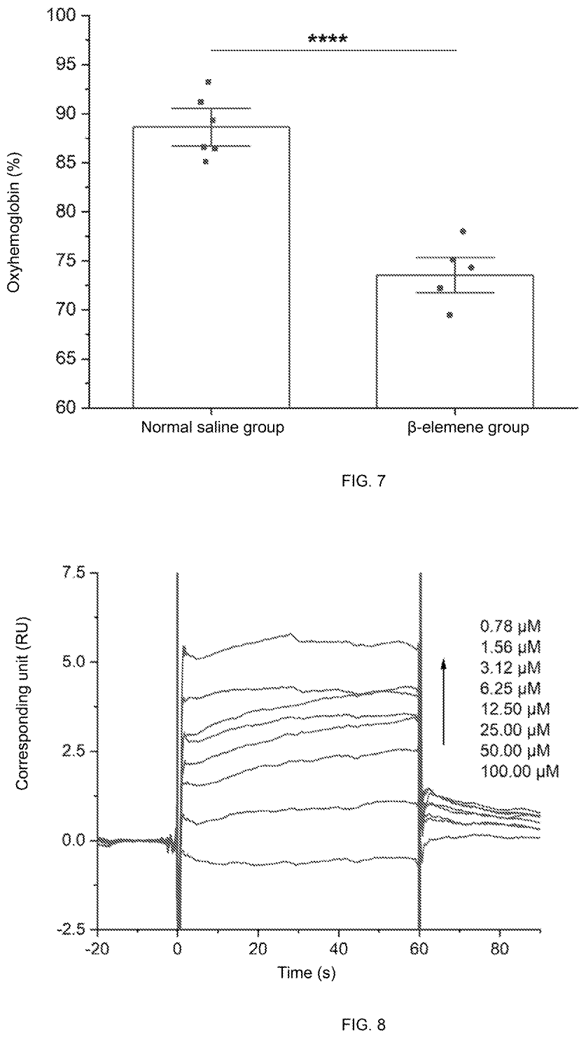

After administration, abdominal aortic blood was taken and immediately analyzed with a blood-gas analyzer. Results are shown in FIGS. 2-7.

After administration, it can be observed that the rats suffered obvious cyanosis of labium, cardiac acceleration, accelerated respiratory rate and increased breathing rate. After stopping administration, the rats had gradually decreased respiratory rate, and gradually reduced breathing rate, and then stabilized to a certain state, indicating that after injecting with BE, the rats suffered dyspnea symptoms, and got recovery automatically after stopping administration.

It can be seen from the results of FIGS. 2-7 that when the rats injected with BE suffered dyspnea, the partial pressure of arterial oxygen and arterial oxygen saturation significantly decrease, indicating that the rats may be in a hypoxia state in vivo. The arterial partial pressure of carbon dioxide significantly decreases while pH has no significant change, indicating that the rats suffer pulmonary overventilation and inadequate oxygen supply, which is consistent with the breathing state of the rats. Total hemoglobin significantly increases, while oxyhemoglobin significantly decreases, suggesting that the symptom is related to the decreased Hb binding to oxygen, thus causing dyspnea in the rats. From animal experiments, it can be seen that dyspnea of the rats induced by BE solution is associated with the binding of Hb to oxygen.

### Example II: Verification of the Interaction Between β-Elemene and Hemoglobin

Surface plasmon resonance (SPR) technology was used to verify the interaction between β-elemene and hemoglobin. SPR may provide affinity for representing the interaction between small molecules and proteins (K_{D} refers to a concentration of an analyte when 50% protein sites on a chip are saturated. The smaller the K_{D} is, the stronger the affinity is, which namely indicates that both are bound with each other more easily). It is generally acknowledged that K_{D} of binding protein to small molecules has an acceptable scope of 10⁻⁴-10⁻⁶M.

1. Preparation of the Analyte

(1) Preparation of a Hb solution: a proper amount of Hb was weighed precisely and prepared into a solution having a concentration of 1 mg/mL with ultrapure water, then 20-fold diluted by pH 4.5 acetic acid for further use;

(2) PBS-P+10× was prepared into PBS-P+2.1× with ultrapure water, then a proper amount of DMSO was taken and prepared into PBS 2× containing 5% DMSO+0.1% Tween 20, and filtered for further use;

(3) A proper amount of BE reference substances were weighed precisely and dissolved into DMSO to be prepared into 10 mM solution, then the solution was diluted with PBS-P+2.1× to a reference substance solution containing 5% DMSO; and then the reference substance solution was diluted successively by 2× PBS-P+ to 100.00 μM, 50.00 μM, 25.00 μM, 12.50 μM, 6.25 μM, 3.12 μM. 1.56 μM and 0.78 μM and vortexed for 1 min, and then filtered by 0.22 μm filter membrane for further use.

2. Preparation of a Calibration Solution

The calibration solution was prepared by the following tables:

| | 4.5% DMSO | 5.8% DMSO |
|---|---|---|
| 2.1 × PBS-P+/1.05 × PBS-P+ | 9.5 mL | 9.5 mL |
| 100% DMSO | 0.45 mL | 0.58 mL |
| Final volume | About 10 mL | About 10 mL |

| | 4.9% DMSO | 5.3% DMSO |
|---|---|---|
| 4.5% DMSO | 0.8 mL | 0.4 mL |
| 5.8% DMSO | 0.4 mL | 0.8 mL |
| Final volume | 1.2 mL | 1.2 mL |

3. Preparation of a Washing Solution

50% DMSO (ultrapure water and filtered DMSO (1:1)) washing solution was prepared to wash needles.

4. Experimental Procedures

(1) Chip activation: carboxyl (channels 1 and 2) on a chip was activated by EDC/NHS;

(2) the channel 2 was selected and coupled to a certain amount of Hb;

(3) the activated carboxyl not coupled to proteins on the chip was blocked by ethanolamine;

(4) a flow channel system was washed by 2×PBS-P+running buffer;

(5) the prepared BE solution and washing solution were put on a shelf successively, and then put on a tray of the shelf for further sample injection;

(6) procedures were set to collect data.

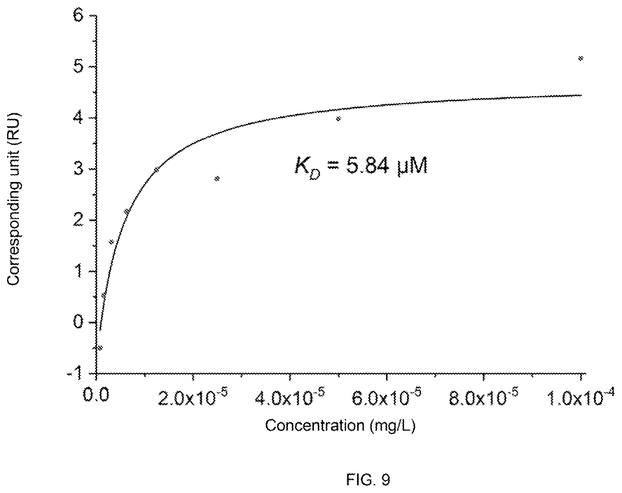

SPR results are shown in FIGS. 8-9. The results shown in FIGS. 8-9 shows that K_{D} of BE and Hb is equal to 5.84 μM, which indicates that there exists stronger affinity between BE and Hb, and there exists intermolecular interaction therebetween as well, namely, BE is bound to Hb easily.

### Example III: Influences of β-Elemene on Hb Structure

A circular dichroism spectrum technology (CD) was used to determine the influences of β-elemene on Hb structure.

Hb mainly presents α-helix at 200-250 nm. If the α-helix structure is changed, so do the shape of CD curve at 200-250 nm. The α-helix content of Hb at 222 nm may be calculated according to the equation (1):

$$α helix \% = \frac{{MRE}_{222} - 2340}{30 , 300} \qquad ( 1 )$$

The mean residue ellipticity (MRE) (degcm²/dmol) is calculated according to the equation (2):

$$MRE = \frac{Observed CD ( mdeg )}{{C}_{p} nl \times 10} \qquad ( 2 )$$

where, Cp is molar concentration (mM) of Hb; n is the number of amino acid residues; l is optical path (mm).

Preparation of a PBS solution: a proper amount of PBS buffer solution was taken and added a small amount of Tween 80, then prepared into a PBS solution containing 0.05% Tween 80.

Preparation of a Hb solution: a proper amount of Hb was weighed precisely and added a proper amount of PBS containing 0.05% Tween 80, then prepared into Hb having a concentration of 30 μM for further use.

Preparation of a BE solution: a proper amount of BE was weighed precisely and added PBS containing 0.05% Tween 80, then prepared into a stock solution having a concentration of 15 μM for further use.

Preparation of a Hb+BE solution: a proper amount of BE and Hb were taken and prepared into a solution having a total volume of 1.5 mL, such that Hb had a final concentration of 5 μM; the BE final concentration was respectively 6.25 μM, 5.00 μM, 2.00 μM, 1.00 μM. 0.

Wavelength detection: 200-250 nm; step size: 0.5 nm; temperature: 25° C.; and response time: 0.5 s.

Operating Steps:

(1) the PBS solution containing 0.05% Tween 80 was first placed into a quartz sample pool having an optical path of 0.1 cm, and the sample pool was put to a sample chamber for detection, and screening was performed for 3 times to obtain a blank curve;

(2) each concentration of sample was placed into a quartz sample pool from small to large (during solution exchange, the sample pool was cleaned by PBS and rinsed with the to-be-detected solution), then the sample pool was put to the sample chamber for detection in turn, and each sample was screened by spectrum for 3 times;

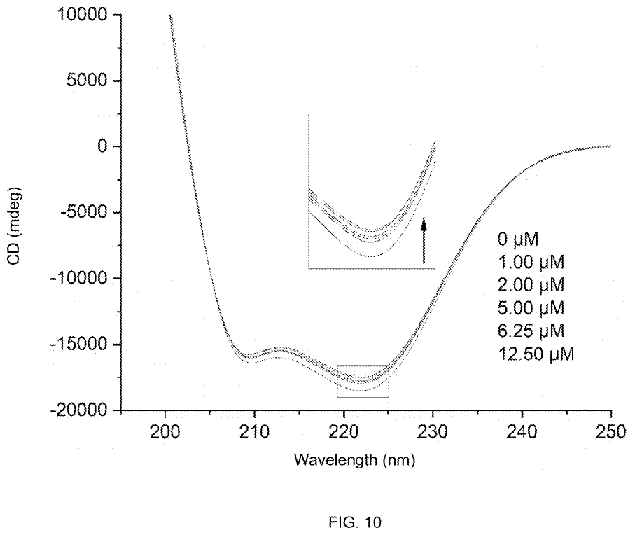

(3) data analysis: the three times of scanning curves for each concentration are averaged, and the blank curve was respectively subtracted for background correction. Results are shown in FIG. 10.

The detection results are shown in FIG. 10. It can be seen from the results as shown in FIG. 10 that with the increase of BE concentration, the curve changes obviously accordingly; that is, BE makes Hb α-helix performing helix removal. The higher the BE concentration is, the more obvious the helix removal effect is. In other words, the secondary structure of Hb is changed by BE, resulting in that Hb may not bind to oxygen, which leads to reduced Hb oxygen-loading amount, such that the organism may not make use of oxygen normally, thereby causing dyspnea.

### Example IV: Evaluation on Adverse Reaction Caused by Sesquiterpenoids in Zedoary Turmeric Oil

The above examples have discussed the mechanism of adverse reaction of dyspnea caused by sesquiterpenoids in zedoary turmeric oil. Based on this, a method for evaluating adverse reaction caused by sesquiterpenoids in zedoary turmeric oil is put forward.

Preparation of Hb solution: a proper amount of Hb was precisely weighed and dissolved by normal saline (ultrasonic treatment for 30 s), and prepared into a solution having a concentration of 2 mg/mL.

Preparation of a BE solution: BE reference substance is precisely weighed and dissolved by 8.8% Tween 80 solution and prepared into a solution having a concentration of 1.5 mg/mL.

Specific Operation:

100 μL Hb solution was added to wells of a 96-well plate, and 100 μL BE sample solution, 100 μL 8.8% Tween 80 solution or 100 μL normal saline (as blank control) were successively added to the wells added with Hb solution. Each sample was repeated with 3 wells; then the microwell plate was covered, and the mixed solution was oscillated for 5 s in an oscillator to be mixed well, standing for 10 min at 25° C. A microplate reader was used for detection, and detection conditions were as follows: spectrum scanning was performed at 37° C. and 230-400 nm with a step size of 5 nm.

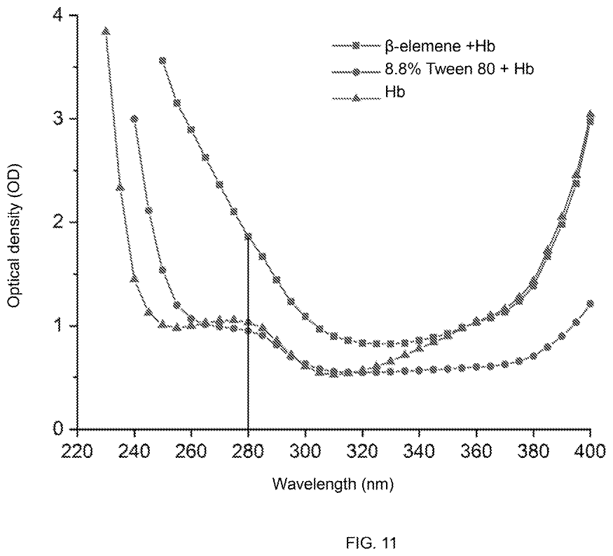

The result curve is shown in FIG. 11.

It can be seen from FIG. 11 that in the presence of BE, Hb ultraviolet absorption peak at about 280 nm is influenced, indicating that there exists effective interaction between aromatic residues (Trp and Tyr) and BE. Moreover, it can be observed that BE makes the Hb absorbance significantly increasing within a scope of 260-300 nm and 8.8% Tween 80 solution (negative control) has basically no interference thereon, which also proves the reliability of the SPR experiment.

From what has been discussed above, a method for evaluating adverse reaction of dyspnea caused by sesquiterpenoids in zedoary turmeric oil is put forward. It is shown by the following formula (3):

$$r = \frac{OD}{{OD}_{Hb}} \qquad ( 3 )$$

Where, OD is an absorbance value of a to-be-detected sample containing sesquiterpenoids in zedoary turmeric oil at a certain concentration at 280 nm after interacting with Hb; OD_{Hb} is an absorbance value of normal saline as a blank control at a wavelength of 280 nm after interacting with Hb; r is a ratio of OD to OD_{Hb}. In this formula, when r>1.5, the result indicates that the sample containing sesquiterpenoids in zedoary turmeric oil at the concentration can bind to Hb, and there is a high risk of dyspnea in clinical use.

For example, as shown in FIG. 11, with regard to the 1.5 mg/mL BE solution, r≈1.8>1.5 indicates that the concentration of BE can bind to Hb, and there exists a high risk of dyspnea in clinical use; for the 8.8% Tween 80 solution, r=0.9<1.5 indicates that the concentration of Tween 80 solution has no obvious effect on Hb, and BE is not influenced at the concentration.

Hereby, the present invention judges the risk of dyspnea in clinical use according to the r value obtained by interacting a medicament or relevant formulation containing sesquiterpenoids in zedoary turmeric oil thereof with Hb, thus achieving the early warning to the adverse reaction of dyspnea caused by sesquiterpenoids in zedoary turmeric oil, or a medicament or relevant formulation containing the component.

What is described above are merely preferred embodiments of the present invention, but the protection scope of the present invention is not limited thereto. Any equivalent replacement or change made by a person skilled in the art based on the technical solution and improvement concept of the present invention within the technical scope disclosed herein shall be covered within the protection scope of the present invention.

What is claimed is:

1. A method for evaluating an adverse reaction of sesquiterpenoids in a zedoary turmeric oil, wherein the method is performed successively according to the following steps:
1) preparing a hemoglobin (Hb) solution and a to-be-determined solution;
2) taking Hb samples from the Hb solution with a same volume and respectively adding the same volume of the to-be-determined solution or normal saline to each of the Hb samples, mixing well and standing;
3) determining an absorbance of each of the Hb samples and the to-be-determined solution with a microplate reader, and comparing the absorbance of the to-be-determined solution with the absorbance of a sample of the normal saline to obtain a value r;
wherein if r>1.5, then a concentration of the to-be-determined solution has a risk of causing dyspnea;
wherein,
$$r = \frac{OD}{{OD}_{Hb}} ,$$
OD is an absorbance at a wavelength of 280 nm the to-be-determined solution after interacting with Hb; OD_{Hb} is an absorbance at a wavelength of 280 nm of the sample of the normal saline after interacting with Hb, wherein the sample of the normal saline acts as a blank control.

2. The method according to claim 1, wherein in the step 1), the Hb solution has a concentration of 2 mg/mL.

3. The method according to claim 1, wherein in the step 2), the same volume of the to-be-determined solution or the normal saline added to each of the Hb samples is 100 μL.

4. The method according to claim 3, wherein the Hb samples has the same volume of 100 μL.

5. The method according to claim 1, wherein the step of mixing well in the step 2) is performed by using an oscillator for oscillation for 5 s.

6. The method according to claim 1, wherein the standing step in the step 2) is performed for 10 min at a condition of 25° C.

7. The method according to claim 1, wherein conditions for determining the absorbance of each of the Hb samples and the to-be-determined solution in the step 3) are as follows: performing a spectrum scanning at 37° C. and 230-400 nm with a step size of 5 nm.
